(19) United States
(12) Patent Application Publication
(10) Pub. No.: US 2025/0322932 A1
(43) Pub. Date: Oct. 16, 2025

(54) METHOD AND KIT OF REDUCING SUGAR INTAKE

(71) Applicant: Ghyas Osman, Wayne, NJ (US)

(72) Inventor: Ghyas Osman, Wayne, NJ (US)

(21) Appl. No.: 19/177,008

(22) Filed: Apr. 11, 2025

Related U.S. Application Data
(60) Provisional application No. 63/634,590, filed on Apr. 16, 2024.
(60) Provisional application No. 63/785,991, filed on Apr. 9, 2025.

(51) Int. Cl. G16H 20/60; A23L 27/30; A23L 33/00; G16H 10/20; G16H 10/60

(52) U.S. Cl. CPC G16H 20/60; A23L 27/34; A23L 33/30; G16H 10/20; G16H 10/60

(57) ABSTRACT

Apparatus and associated methods relate to a system and method of reducing sugar-intake amounts including gradually transitioning from intaking sugar to intaking monk fruit sweetener with erythritol. In an illustrative example, the system of reducing sugar-intake may include a kit with a set of predetermined sugar and monk fruit sweetener with erythritol blends. The kit may, for example, include a dietary schedule configured to gradually transition a user of the kit from sugar to monk fruit sweetener with erythritol over a predetermined period. The kit may, for example, include an evaluation engine configured to determine a user's a baseline sugar-intake level and periodically, at predetermined times, evaluate the user's response to transitioning from sugar to monk fruit sweetener with erythritol. Various embodiments may advantageously gradually decrease a person's sugar-intake levels.

### CROSS-REFERENCE TO RELATED APPLICATIONS

This application is a non-provisional application and claims the benefit of U.S. Application Ser. No. 63/634,590, titled “METHOD AND KIT OF REDUCING SUGAR INTAKE,” filed by Ghyas Osman on Apr. 16, 2024.

This application also claims the benefit of U.S. Provisional Application Ser. No. 63/785,991 titled “METHOD AND KIT OF REDUCING SUGAR INTAKE,” filed by Ghyas Osman on Apr. 9, 2025.

This application incorporates the entire contents of the foregoing applications herein by reference.

### TECHNICAL FIELD

Various embodiments relate generally to dieting and healthcare.

### BACKGROUND

Sugar is a type of carbohydrate that serves as a primary source of energy for the body. It occurs naturally in fruits, vegetables, and dairy products in forms such as glucose, fructose, and lactose. However, refined sugars, such as sucrose from sugarcane, are widely used in processed foods and beverages. While sugar enhances flavor and provides quick energy, excessive consumption has been linked to health concerns such as obesity, diabetes, and tooth decay.

Reducing sugar intake may, for example, lead to significant health benefits, including lower risk of obesity, type 2 diabetes, and heart disease, as reducing sugar consumption decreases the number of empty calories and insulin spikes in one's diet. Alternatives to sugar, such as, erythritol and monk fruit sweetener, may, for example, offer the sweetness without the adverse effects on health, making them suitable for those looking to reduce sugar intake while still enjoying sweet flavors. These alternatives vary in taste and nutritional profile, offering options for different dietary needs and preferences.

### SUMMARY

Apparatus and associated methods relate to a system and method of reducing sugar-intake amounts including gradually transitioning from intaking sugar to intaking monk fruit sweetener with erythritol. In an illustrative example, the system of reducing sugar-intake may include a kit with a set of predetermined sugar and monk fruit sweetener with erythritol blends. The kit may, for example, include a dietary schedule configured to gradually transition a user of the kit from sugar to monk fruit sweetener with erythritol over a predetermined period. The kit may, for example, include an evaluation engine configured to determine a user's a baseline sugar-intake level and periodically, at predetermined times, evaluate the user's response to transitioning from sugar to monk fruit sweetener with erythritol. Various embodiments may advantageously gradually decrease a person's sugar-intake levels.

Various embodiments may achieve one or more advantages. For example, some embodiments may advantageously wean a person off sugar in one to two months. Some implementations may, for example, advantageously reduce a person's A1C (hemoglobin A1c) levels. Some embodiments may, for example, reduce caloric intake from sugar consumption by up to 75%.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary sugar-intake reduction system and kit employed in an illustrative use-case scenario.

FIG. 2 depicts a schematic of an exemplary sugar-intake reduction kit.

FIG. 3 depicts an exemplary dietary schedule.

FIG. 4 is a block diagram depicting an exemplary application data store on an app-enabled device.

FIG. 5 depicts an exemplary method of reducing sugar intake utilizing a kit and an online application.

FIG. 6 depicts an exemplary feedback method loop of a dietary schedule.

Like reference symbols in the various drawings indicate like elements.

### DETAILED DESCRIPTION OF ILLUSTRATIVE EMBODIMENTS

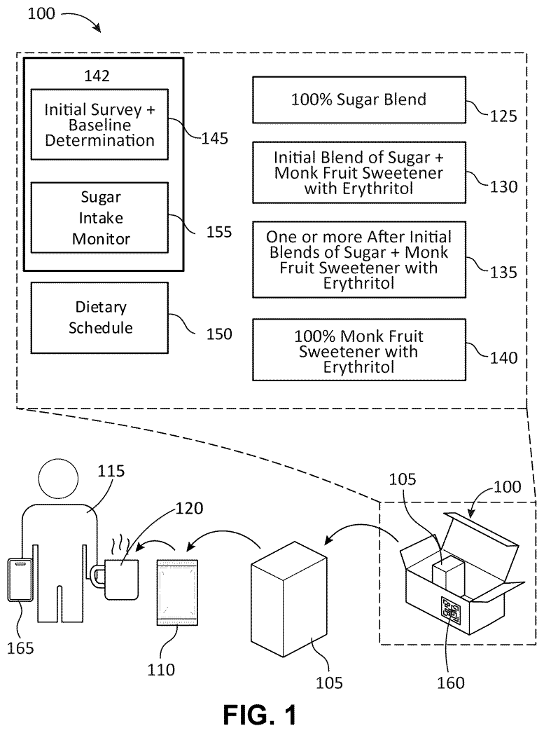

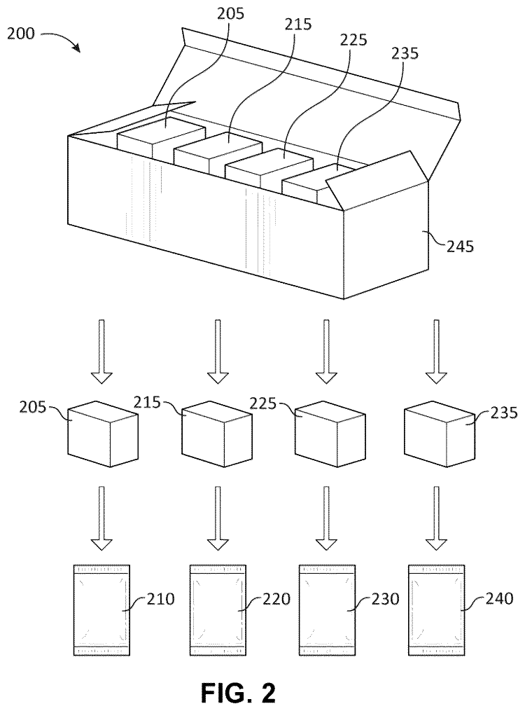

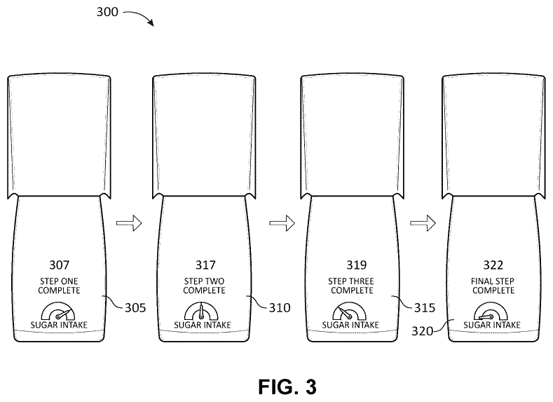

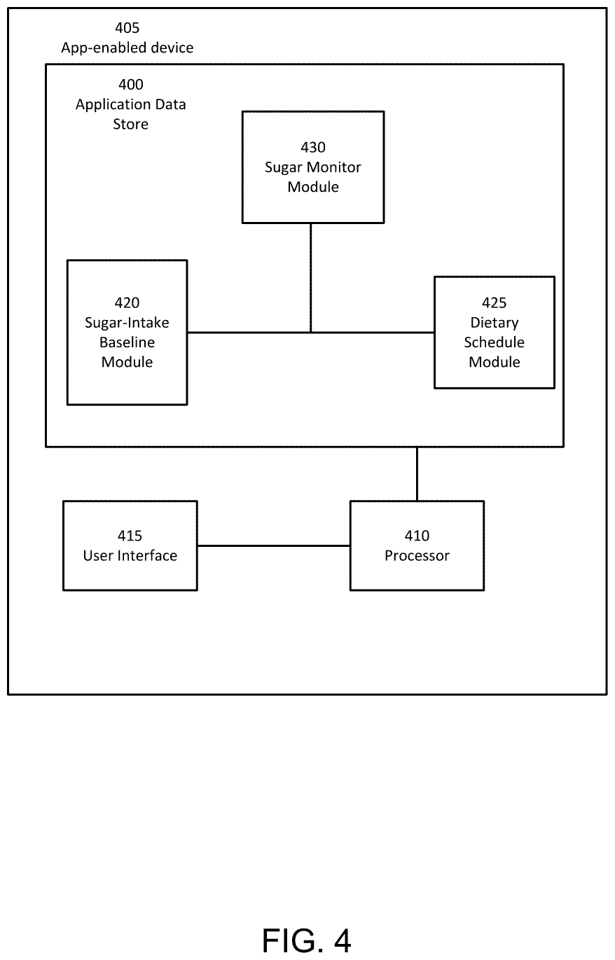

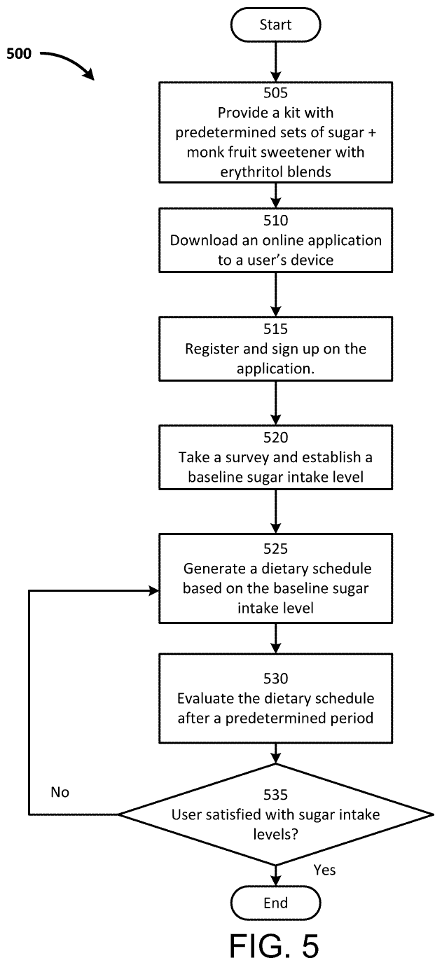

To aid understanding, this document is organized as follows. First, to help introduce discussion of various embodiments, a sugar-intake reduction system is introduced with reference to FIG. 1. Second, that introduction leads into a description with reference to FIG. 2 of an exemplary embodiments of a sugar-intake reduction kit. Third, with reference to FIG. 3 is described an exemplary dietary schedule. Fourth, with reference to FIG. 4, the discussion turns to a block diagram of an exemplary application data store on an app-enabled device. Fifth, and with reference to FIG. 5, this document describes an exemplary method of reducing sugar intake utilizing a kit and an online application. Sixth, this document describes an exemplary feedback method loop of a dietary schedule. Finally, the document discusses further embodiments, exemplary applications and aspects relating to a sugar-intake reduction system.

FIG. 1 depicts an exemplary sugar-intake reduction system and kit 100 employed in an illustrative use-case scenario. The kit 100 includes one or more containers 105. The one or more containers 105 include one or more packages 110. The one or more packages 110 may, for example, include a predetermined blend of sugar and monk fruit sweetener with erythritol that may, for example, act as an artificial sweetener for a user's 115 dietary intake 120. The user's 115 dietary intake 120 may, for example, include a drink in a cup, as depicted.

The kit 100 may, for example, be employed by the user 115 to advantageously reduce the user's 115 sugar-intake levels. The kit 100 may, for example, provide a method by which the user 115 may, for example, reduce sugar-intake levels. The kit 100 may, for example, advantageously wean the user 115 off of sugar to monk fruit sweetener with erythritol.

The one or more packages 110 may, for example, include an at least one 100% sugar package 125. The at least one 100% sugar package 125 includes a blend of sugar and monk fruit sweetener with erythritol that includes 100% sugar and 0% monk fruit sweetener with erythritol. By way of example, and not limitation, sugar may include granulated sugar. By way of example, and not limitation, sugar may include brown sugar. By way of example, and not limitation, sugar may include cane sugar. By way of example, and not limitation, sugar may include beet sugar. By way of example, and not limitation, sugar may include powdered sugar. By way of example, and not limitation, sugar may include molasses. By way of example, and not limitation, sugar may include raw sugar. By way of example, and not limitation, sugar may include high-fructose corn syrup.

The one or more packages 110 includes an at least one initial blend package 130. The at least one initial blend package 130 may, for example, include a blend of 90% sugar and 10% monk fruit sweetener with erythritol. The at least one initial blend package 130 may, for example, include a blend of 75% sugar and 25% monk fruit sweetener with erythritol.

The one or more packages 110 includes one or more after initial blend packages 135. The one or more after initial blend packages 135 may, for example, include a blend of 80% sugar and 20% monk fruit sweetener with erythritol. The one or more after initial blend packages 135 may, for example, include a blend of 75% sugar and 25% monk fruit sweetener with erythritol. The one or more after initial blend packages 135 may, for example, include a blend of 70% sugar and 30% monk fruit sweetener with erythritol. The one or more after initial blend packages 135 may, for example, include a blend of 60% sugar and 40% monk fruit sweetener with erythritol. The one or more after initial blend packages 135 may, for example, include a blend of 50% sugar and 50% monk fruit sweetener with erythritol. The one or more after initial blend packages 135 may, for example, include a blend of 40% sugar and 60% monk fruit sweetener with erythritol. The one or more after initial blend packages 135 may, for example, include a blend of 30% sugar and 70% monk fruit sweetener with erythritol. The one or more after initial blend packages 135 may, for example, include a blend of 25% sugar and 75% monk fruit sweetener with erythritol. The one or more after initial blend packages 135 may, for example, include a blend of 20% sugar and 80% monk fruit sweetener with erythritol. The one or more after initial blend packages 135 may, for example, include a blend of 10% sugar and 90% monk fruit sweetener with erythritol.

The one or more packages 110 includes at least one 100% monk fruit with erythritol packages 140. The at least one 100% monk fruit sweetener with erythritol packages 140 includes a blend of sugar and monk fruit sweetener with erythritol that includes 0% sugar and 100% monk fruit sweetener with erythritol.

The at least one initial blend package 130, one or more after initial blend packages 135, and at least one 100% monk fruit sweetener with erythritol packages 140 may, for example, provide a set of predetermined sugar and monk fruit sweetener with erythritol blends.

The kit 100 includes an evaluation engine 142. The evaluation engine may, for example, include a method of weight loss. The evaluation engine may, for example, transition from sugar to monk fruit sugar. The evaluation engine 142 includes an initial baseline determination survey 145 configured to determine a user's 115 baseline sugar intake amount. Establishing a user's 115 baseline sugar-intake amount may, for example, start with the initial baseline determination survey 145 to capture the user's current dietary habits and health status.

The kit 100 includes a dietary schedule 150 configured to gradually transition the user 115 of the kit 100 from sugar to monk fruit sweetener with erythritol. The dietary schedule 150 may, for example, be based on the determined baseline sugar intake amount of the user 115. The dietary schedule 150 may, for example, provide a schedule including predetermined periods at which to transition sugar-intake amounts. For example, a dietary schedule 150 may include a predetermined period of one month in which the user 115 intakes sugar and monk fruit sweetener with erythritol blends provided within the at least one initial blend package 130. After the one month, the dietary schedule 150 may, for example, include a transition in which the user 115 intakes sugar and monk fruit sweetener with erythritol blends provided within the one or more after initial blend packages 135 for a predetermined period of one month. By way of example, and not limitation, the predetermined period may, for example, include a period of two months. By way of example, and not limitation, the predetermined period may, for example, include a period of three months.

The evaluation engine 142 includes a sugar intake monitor 155 configured to periodically, at predetermined times, evaluate the user's 115 response to transitioning from sugar to monk fruit sweetener with erythritol. The predetermined time of evaluation may, for example, include a period of two weeks. The predetermined time of evaluation may, for example, include a period of three weeks. The sugar intake monitor 155 may, for example, provide a survey with questions regarding the user's satisfaction with the dietary schedule 150. The sugar intake monitor 155 may, for example, determine the user's 115 satisfaction with the dietary schedule 150 based on the user's 115 answers to the survey. The dietary schedule 150 may, for example, adjust based on the determined satisfaction level.

The evaluation engine 142 may, for example, evaluate the user's physiological response to the dietary transition. The evaluation engine 142 may, for example, evaluate the user's 115 physiological response to the dietary transition. The evaluation engine 142 may, for example, evaluate the user's 115 behavioral response to the dietary transition. The evaluation engine 142 may, for example, form evaluation outcomes based on the physiological and behavioral responses. The evaluation outcomes may, for example, include surveying a user's 115 weight. The evaluation outcomes may, for example, include surveying a user's 115 insulin sensitivity. The evaluation outcomes may, for example, include surveying a user's 115 energy levels. The evaluation outcomes may, for example, include surveying a ranking of a user's 115 cravings for sugar. The evaluation outcomes may, for example, include surveying a user's diet; a survey of a user's 115 mood changes. The evaluation outcomes may, for example, include surveying of a user's 115 adherence to the dietary schedule. The evaluation outcomes may, for example, include surveying a perceived satisfaction with the sweetener monk fruit erythritol blend. The dietary schedule 150 may, for example, adjust based on the evaluation outcome.

The kit 100 may, for example, include a visual data marker 160. The visual data marker 160 may, for example, include a quick response (QR) code. The user 115 may, for example, have a computing device 165 that may, for example, scan the visual data marker 160. The computing device may, for example, include a mobile phone. Scanning the visual data marker 160 may, for example, direct the computing device 165 to download an online application (not depicted). As will be explained in more detail with reference to FIG. 4, the online application may, for example, provide the evaluation engine 142, the initial baseline determination survey 145, the dietary schedule 150, and the sugar intake monitor 155.

FIG. 2 depicts a schematic of an exemplary sugar-intake reduction kit 200. The sugar-intake reduction kit 200 includes an initial container 205. The initial container 205 includes one or more initial packages 210. The one or more initial packages 210 may, for example, include an initial blend of sugar and monk fruit sweetener with erythritol. The initial blend of sugar and monk fruit sweetener with erythritol may, for example, be arranged in a substantially similar manner to the blend of sugar and monk fruit sweetener with erythritol within the at least one initial blend package 130. A user of the kit 200 may, for example, advantageously start transitioning from sugar to monk fruit sweetener with erythritol through intaking the blend of sugar and monk fruit sweetener within the one or more initial packages 210 instead of intaking sugar.

The sugar-intake reduction kit 200 includes one or more after initial containers 215 and 225. The one or more after initial containers 215 and 225 includes one or more after initial packages 220 and 230. The one or more after initial packages 220 and 230 may, for example, include a blend of sugar and monk fruit sweetener with erythritol. The blend of sugar and monk fruit sweetener with erythritol may, for example, be arranged in a substantially similar manner to the blend of sugar and monk fruit sweetener with erythritol within the one or more after initial blend packages 135. A user of the kit 200 may, for example, advantageously continue transitioning from sugar to monk fruit sweetener with erythritol through intaking the blend of sugar and monk fruit sweetener within the after initial packages 220 and 230 after intaking the one or more initial packages 210 over a predetermined period.

The sugar-intake reduction kit 200 includes at least one 100% monk fruit sweetener with erythritol containers 235. The at least one 100% monk fruit sweetener with erythritol containers 235 includes at least one 100% monk fruit sweetener with erythritol packages 240. The at least one 100% monk fruit sweetener with erythritol packages 240 may, for example, include a blend of sugar and monk fruit sweetener with erythritol. The blend of sugar and monk fruit sweetener with erythritol may, for example, be arranged in a substantially similar manner to the blend of sugar and monk fruit sweetener with erythritol within the at least one 100% monk fruit sweetener with erythritol packages 140. A user of the kit 200 may, for example, advantageously finish transitioning from sugar to monk fruit sweetener with erythritol through intaking the blend of sugar and monk fruit sweetener within the at least one 100% monk fruit sweetener with erythritol packages 240 after one or more after initial packages 220 and 230 over a predetermined period.

The sugar-intake reduction kit 200 includes a visual data marker 245. The visual data marker 245 may, for example, be arranged in substantially similar manner to the visual data marker 160.

FIG. 3 depicts an exemplary dietary schedule 300. The dietary schedule 300 includes an initial container 305. The initial container 305 may, for example, include one or more initial packages (not depicted). The one or more initial packages may, for example, include an initial blend of sugar and monk fruit sweetener with erythritol. The initial blend of sugar and monk fruit sweetener with erythritol in the one or more initial packages may, for example, be arranged in a substantially similar manner to the blend of sugar and monk fruit sweetener with erythritol within the at least one initial blend package 130. The initial container 305 may, for example, include a written message 307 indicating the period of intaking the initial blend of sugar and monk fruit sweetener with erythritol is over. The written message 307 may, for example, state “step one complete.”

The dietary schedule 300 includes one or more after initial containers 310 and 315. The one or more after initial containers 310 and 315 may, for example, include one or more after initial packages (not depicted). The one or more after initial packages may, for example, include an after initial blend of sugar and monk fruit sweetener with erythritol. The after initial blend of sugar and monk fruit sweetener with erythritol in the one or more initial packages may, for example, be arranged in a substantially similar manner to the blend of sugar and monk fruit sweetener with erythritol within the one or more after initial blend packages 135. The one or more after initial containers 310 and 315 may, for example, include a written message 317 and 319 indicating the period of intaking the after initial blend of sugar and monk fruit sweetener with erythritol is over. The written message 317 and 319 may, for example, state “step two complete.” The written message 317 may, for example, state “step three complete.”

The dietary schedule 300 includes an at least one 100% monk fruit sweetener with erythritol container 320. The at least one 100% monk fruit sweetener with erythritol containers 320 may, for example, include an at least one 100% monk fruit sweetener with erythritol packages (not depicted). The at least one 100% monk fruit sweetener with erythritol packages may, for example, include a blend of 100% monk fruit sweetener with erythritol. The at least one 100% monk fruit sweetener with erythritol container 320 may, for example, include a written message 322 indicating a transition from sugar to 100% monk fruit sweetener with erythritol. The written message 322 may, for example, state “final step complete.”

FIG. 4 is a block diagram depicting an exemplary application data store 400 on an app-enabled device 405. The application data store 400 may, for example, provide an online application that may, for example, be downloaded on an app-enabled device 405. The app-enabled device 405 may, for example, include a mobile phone. The app-enabled device 405 includes a processor 410. The processor 410 couples a user interface 415. The processor 410 couples the application data store 400.

The application data store 400 includes a sugar-intake baseline module 420. The sugar-intake baseline module 420 may, for example, generate a survey with questions regarding a baseline sugar-intake level. The processor 410 may, for example, display the survey on the user interface 415 such that a user of the app-enabled device may, for example, answer the questions to the survey. The processor 410 may, for example, retrieve the answers to the survey and transmit the answers to the sugar-intake module 420. The sugar-intake module 420 may, for example, determine a baseline sugar-intake level based on the answers to the questions from the retrieved answered survey. The processor 410 may, for example, retrieve the baseline sugar-intake level and transmit the baseline sugar-intake level to a dietary schedule module 425. Based on the retrieved baseline sugar-intake level, the dietary schedule module 425 may, for example, generate a dietary schedule configured to gradually transition a user from sugar to monk fruit sweetener with erythritol over a predetermined period, such as the embodiments described with reference to the dietary schedule 150 and 300.

The application data store 400 includes a sugar monitor module 430. The sugar monitor module 430 may, for example, generate a survey with questions regarding a user's satisfaction with the dietary schedule. The processor 410 may, for example, display the survey on the user interface 415 such that a user of the app-enabled device may, for example, answer the questions to the survey. The processor 410 may, for example, retrieve the answers to the survey and transmit the answers to the sugar monitor module 430. The sugar monitor module 430 may, for example, determine a satisfaction level based on the answers to the questions from the retrieved answered survey. The processor 410 may, for example, retrieve the satisfaction level and transmit the satisfaction level to a dietary schedule module 425. Based on the retrieved baseline sugar-intake level, the dietary schedule module 425 may, for example, generate an adjusted dietary schedule configured to gradually transition a user from sugar to monk fruit sweetener with erythritol over a predetermined period.

FIG. 5 depicts an exemplary method 500 of reducing sugar intake utilizing a kit and an online application. In a step 505, a kit, such as the embodiments described with reference to the kit 100 and 200, including predetermined sets of sugar and monk fruit sweetener with erythritol blends is provided to a user. The predetermined sets of sugar and monk fruit sweetener with erythritol blends may, for example, include different proportions of sugar and monk fruit sweetener with erythritol blends, such as those proportions described with reference to and provided within the at least one initial blend package 130, the one or more after initial blend packages 135, and the at least one 100% monk fruit sweetener with erythritol packages 140. The kit may, for example, include a visual data marker, such as the embodiments described with reference to visual data marker 160. The visual data marker may, for example, include a QR code.

In a step 510, the user downloads an online application to the user's device. The user's device may, for example, include a mobile phone. For example, the user may scan the QR code with the mobile phone such that the mobile phone may be directed to download the online application, for example, such as the embodiments described with reference to the application data store 400.

In a step 515, the user registers and signs up on the application. For example, the user may provide an email address and a password such that the user creates a profile on the app. In a step 520, the online application generates a survey, such as, for example, the survey generated as described with reference to the sugar-intake baseline module 420. The user takes the survey to establish a baseline sugar intake level.

In a step 525, based on the established baseline sugar intake level, the online application generates a dietary schedule configured to gradually transition the user from sugar to monk fruit sweetener with erythritol, such as, for example, the survey generated as described with reference to the dietary schedule module 425. The dietary schedule may, for example, include predetermined periods at which the user intakes the predetermined sets of sugar and monk fruit sweetener with erythritol blends. For example, the dietary schedule may include a predetermined period instructing the user to intake the blends within the at least one initial blend package 130. At the end of the predetermined period, the dietary schedule may, for example, instructs the user to intake the blends in the one or more after initial blend packages 135. The user may, for example, follow the dietary schedule to advantageously transition from intaking sugar to intaking monk fruit sweetener with erythritol.

In a step 530, the online application evaluates the user's satisfaction with the dietary schedule after the user has been on the dietary scheduled for a predetermined period. For example, the online application may generate a survey with questions regarding a user's satisfaction, such as, for example, as described with reference to the sugar monitor module 430. The user may, for example, take the survey such that the online application may, for example, determine the user's satisfaction level with the dietary schedule.

In a step 535, a decision point is reached where the online application, based on the determined user satisfaction level, determines whether a user is satisfied with the dietary schedule. If the online application determines a user is satisfied with the dietary schedule, then the method 500 ends, and the user remains on the dietary schedule. If the online application determines a user is not satisfied with the dietary schedule, the method 500 reverts to step 525 to generate a new dietary schedule.

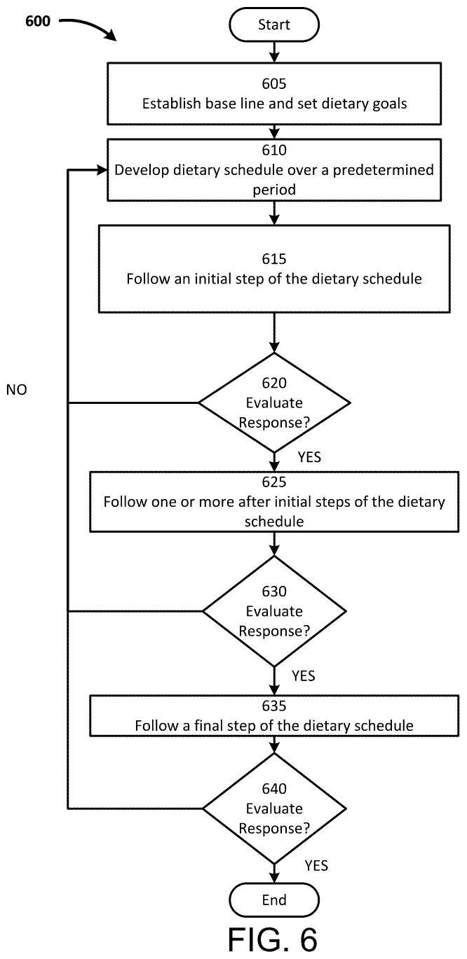

FIG. 6 depicts an exemplary feedback method loop 600 of a dietary schedule. In a step 605, user of the method establishes a sugar-intake baseline and sets dietary goals, such as caloric intake and weight loss goals, providing a framework for personalized nutrition management.

In a step 610, a dietary schedule, for example, such as the embodiments described with reference to the dietary schedule 150 and 300, is developed. The dietary schedule may, for example, incorporate a gradual increase of monk fruit sweetener with erythritol and a gradual decrease of sugar over a predetermined period.

In a step 615, the user of the feedback method loop 600 follows an initial step of the dietary schedule. For example, the initial step may include intaking the blends of sugar and monk fruit sweetener with erythritol provided within the at least one initial blend package 130 over a period of one month.

Following this, in step 620, there is an evaluation of the user's response to the blend, which informs whether to proceed with the dietary schedule. If the evaluation is negative, the feedback method loop 600 reverts to step 610. If the evaluation is positive in step 620, the user of the feedback method loop 600 progresses to a step 625.

In step 625, the user progresses to follow one or more after initial steps of the dietary schedule. For example, the one or more after initial steps may include intaking the blends of sugar and monk fruit sweetener with erythritol provided within one or more after initial blend packages 135 over a period of one month. In step 625, the dietary schedule may, for example, instruct the user to intake less sugar and more monk fruit with erythritol sweetener than the user was instructed to take in step 615.

Following this, in step 630, there is an evaluation of the user's response to the blend, which informs whether to proceed with the dietary schedule. If the evaluation is negative, the feedback method loop 600 reverts to step 610. If the evaluation is positive in step 630, the user of the feedback method loop 600 progresses to a step 635.

In step 635, the user progresses to follow one or more after initial steps of the dietary schedule. For example, the one or more after initial steps may include intaking the blends of sugar and monk fruit sweetener with erythritol provided within at least one 100% monk fruit sweetener with erythritol packages 140 over a period of one month. In step 635, the dietary schedule may, for example, instruct the user to intake blends containing 100% monk fruit with erythritol sweetener.

Following this, in step 640, there is an evaluation of the user's response to the blend, which informs whether to proceed with the dietary schedule. If the evaluation is negative, the feedback method loop 600 reverts to step 610. If the evaluation is positive in step 640, the feedback method loop 600 ends and the user remains on the dietary schedule.

Although various embodiments have been described with reference to the figures, other embodiments are possible.

Although an exemplary system has been described with reference to FIGS. 1-6, other implementations may be deployed in other industrial, scientific, medical, commercial, and/or residential applications.

In industrial applications, the method of reducing sugar intake may, for example, be used in food processing and manufacturing industries. By incorporating monk fruit sugar blends into various products, manufacturers may, for example, create lower-sugar alternatives without compromising taste, meeting consumer preferences for healthier choices.

Scientifically, this method could be studied and analyzed in nutrition research to understand its impact on metabolic health and disease prevention. Researchers may, for example, conduct clinical trials to evaluate the effectiveness of sugar reduction strategies using monk fruit sugar blends, providing insights into dietary interventions for combating obesity, diabetes, and other related conditions.

In medical settings, healthcare professionals may, for example, utilize this method as part of dietary counseling and intervention programs for patients with conditions such as obesity, diabetes, and metabolic syndrome. By prescribing sugar-reducing kits and guiding patients through gradual transitions to monk fruit sugar blends, healthcare providers can support long-term lifestyle changes that promote better health outcomes.

Commercially, food and beverage companies may, for example, market monk fruit sugar blends as premium ingredients in their products, catering to health-conscious consumers seeking alternatives to traditional sugars. The monk fruit blend may, for example, creates opportunities for new product development and market differentiation, tapping into the growing trend of clean label and natural sweeteners.

In residential settings, individuals and families may, for example, adopt this method to improve their dietary habits and overall well-being. By substituting traditional sugars with monk fruit sugar blends in home cooking and baking, households may, for example, reduce their sugar intake without sacrificing flavor, leading to healthier eating habits for the households' members. This approach may, for example, be used to align the user's increasing awareness of the importance of nutrition and maintaining good health at the individual level.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as a 9V (nominal) batteries, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device. The display device may, for example, include an LED (light-emitting diode) display. In some implementations, a display device may, for example, include a CRT (cathode ray tube). In some implementations, a display device may include, for example, an LCD (liquid crystal display). A display device (e.g., monitor) may, for example, be used for displaying information to the user. Some implementations may, for example, include a keyboard and/or pointing device (e.g., mouse, trackpad, trackball, joystick), such as by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the modules may involve execution of preprogrammed instructions, software executed by a processor, or some combination thereof. For example, various modules may involve both hardware and software.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A system of reducing sugar intake comprising:
a predetermined sugar and monk fruit erythritol sweetener blends kit comprising:
a plurality of predetermined sugar and monk fruit erythritol sweetener blends comprising:
a blend of 90% sugar and 10% monk fruit erythritol sweetener blend;
a blend of 75% sugar and 25% monk fruit erythritol sweetener blend;
a blend of 50% sugar and 50% monk fruit erythritol sweetener blend;
a blend of 25% sugar and 75% monk fruit erythritol sweetener; and,
a 100% monk fruit erythritol sweetener blend;
a dietary schedule configured to gradually transition a user of the kit from sugar to monk fruit erythritol sweetener, over a predetermined period comprising:
administering the blend of 90% sugar and 10% monk fruit erythritol sweetener blend;
administering the blend of 75% sugar and 25% monk fruit erythritol sweetener blend;
administering the blend of 50% sugar and 50% monk fruit erythritol sweetener blend;
administering the blend of 25% sugar and 75% monk fruit erythritol sweetener blend; and,
administering the 100% monk fruit erythritol sweetener blend; and
an evaluation engine configured to periodically determine a baseline sugar-intake evaluating the user's response to transitioning from sugar to monk fruit erythritol sweetener, the evaluation engine operations comprising:
determining a baseline sugar-intake level of the user;
evaluating the user's physiological response to the dietary transition;
evaluating the user's behavioral response to the dietary transition;
forming evaluation outcomes based on the physiological and behavioral responses comprising:
surveying a user's weight;
surveying user's insulin sensitivity;
surveying a user's energy levels;
surveying a ranking of a user's cravings for sugar;
surveying a user's diet;
surveying of a user's mood changes;
surveying of a user's adherence to the dietary schedule; and,
surveying a perceived satisfaction with the sweetener monk fruit erythritol blend; and,
adjusting the dietary schedule based on the evaluation outcome.

2. The system of reducing sugar intake of claim 1, wherein the plurality of predetermined sugar and monk fruit erythritol sweetener blends further comprises:
80% sugar and 20% monk fruit erythritol sweetener blend;
70% sugar and 30% monk fruit erythritol sweetener blend;
60% sugar and 40% monk fruit erythritol sweetener blend;
40% sugar and 60% monk fruit erythritol sweetener blend;
30% sugar and 70% monk fruit erythritol sweetener blend;
20% sugar and 80% monk fruit erythritol sweetener blend; and,
10% sugar and 90% monk fruit erythritol sweetener blend

3. The system of reducing sugar intake of claim 2, wherein the dietary schedule is configured to gradually transition a user of the kit from sugar to monk fruit erythritol sweetener over a predetermined period comprising:
administering the 80% sugar and 20% monk fruit erythritol sweetener blend;
administering the 70% sugar and 30% monk fruit erythritol sweetener blend;
administering the 60% sugar and 40% monk fruit erythritol sweetener blend;
administering the 40% sugar and 60% monk fruit erythritol sweetener blend;
administering the 30% sugar and 70% monk fruit erythritol sweetener blend;
administering the 20% sugar and 80% monk fruit erythritol sweetener blend; and,
administering the 10% sugar and 90% monk fruit erythritol sweetener blend.

4. The system of reducing sugar intake of claim 1, wherein the predetermined period further comprises a period of one month.

5. The system of reducing sugar intake of claim 1, wherein the predetermined period further comprises a period of two months.

6. The system of reducing sugar intake of claim 1, wherein a periodic time of evaluation comprises a period of two weeks.

7. The system of reducing sugar intake of claim 1, wherein a periodic time of evaluation comprises a period of three weeks.

8. A system of reducing sugar intake comprising:
a predetermined sugar and monk fruit erythritol sweetener blends kit comprising:
a plurality of predetermined sugar and monk fruit erythritol sweetener blends comprising:
a blend of 90% sugar and 10% monk fruit erythritol sweetener blend;
a blend of 75% sugar and 25% monk fruit erythritol sweetener blend;
a blend of 50% sugar and 50% monk fruit erythritol sweetener blend;
a blend of 25% sugar and 75% monk fruit erythritol sweetener; and,
a 100% monk fruit monk fruit erythritol sweetener blend;
a dietary schedule configured to gradually transition a user of the kit from sugar to monk fruit erythritol sweetener, over a predetermined period comprising:
administering the blend of 90% sugar and 10% monk fruit erythritol sweetener blend;
administering the blend of 75% sugar and 25% monk fruit erythritol sweetener blend;
administering the blend of 50% sugar and 50% monk fruit erythritol sweetener blend;
administering the blend of 25% sugar and 75% monk fruit erythritol sweetener blend; and,
administering the 100% monk fruit sweetener with erythritol blend; and
an evaluation engine configured to periodically determine a baseline sugar-intake evaluating the user's response to transitioning from sugar to monk fruit erythritol sweetener, the evaluation engine operations comprising:
determining a baseline sugar-intake level of the user;
evaluating the user's physiological response to the dietary transition;
evaluating the user's behavioral response to the dietary transition;
forming evaluation outcomes based on the physiological response and behavioral response; and,
adjusting the dietary schedule based on the evaluation outcome.

9. The system of reducing sugar intake of claim 8, wherein the evaluation engine further comprises an application data store comprising a program of instructions; and a processor operably coupled to the application data store such that, when the processor executes the program of instructions, the processor causes operations to be performed to an app-enabled device, the operations comprising:
generating a survey with questions regarding a baseline sugar-intake level;
retrieving an answered survey regarding a baseline sugar-intake level;
determining a baseline sugar-intake level based on the retrieved answered survey; and,
generating a dietary schedule based on the determined baseline sugar-intake level.

10. The system of reducing sugar intake of claim 9, wherein the operations further comprise:
generating a survey with questions regarding satisfaction with the dietary schedule;
retrieving an answered survey regarding satisfaction with the dietary schedule;
determining a satisfaction level based on the retrieved answered survey; and,
generating a dietary schedule based on the determined satisfaction level.

11. The system of reducing sugar intake of claim 8, wherein the plurality of predetermined sugar and monk fruit erythritol sweetener blends further comprises:
80% sugar and 20% monk fruit erythritol sweetener blend;
70% sugar and 30% monk fruit erythritol sweetener blend;
60% sugar and 40% monk fruit erythritol sweetener blend;
40% sugar and 60% monk fruit erythritol sweetener blend;
30% sugar and 70% monk fruit erythritol sweetener blend;
20% sugar and 80% monk fruit erythritol sweetener blend; and,
10% sugar and 90% monk fruit erythritol sweetener blend

12. The system of reducing sugar intake of claim 11, wherein the dietary schedule is configured to gradually transition a user of the kit from sugar to monk fruit erythritol sweetener over a predetermined period comprising:
administering the 80% sugar and 20% monk fruit erythritol sweetener blend;
administering the 70% sugar and 30% monk fruit erythritol sweetener blend;
administering the 60% sugar and 40% monk fruit erythritol sweetener blend;
administering the 40% sugar and 60% monk fruit erythritol sweetener blend;
administering the 30% sugar and 70% monk fruit erythritol sweetener blend;
administering the 20% sugar and 80% monk fruit erythritol sweetener blend; and,
administering the 10% sugar and 90% monk fruit erythritol sweetener blend.

13. The system of reducing sugar intake of claim 8, wherein the predetermined period further comprises a period of one month.

14. The system of reducing sugar intake of claim 8, wherein the predetermined period further comprises a period of two months.

15. The system of reducing sugar intake of claim 8, wherein the predetermined period further comprises a period of three months.

16. The system of reducing sugar intake of claim 8, wherein a periodic time of evaluation comprises a period of two weeks.

17. The system of reducing sugar intake of claim 8, wherein a periodic time of evaluation comprises a period of three weeks.

18. The system of reducing sugar intake of claim 8, wherein the evaluation engine further comprises adjusting the dietary schedule based on the application of use of the predetermined sugar and monk fruit erythritol sweetener blends.

19. The system of reducing sugar intake of claim 8, wherein evaluating the physiological response comprises the collection of data concerning: a survey of the user's weight; a survey of the user's insulin sensitivity; and, a survey of the user's energy levels.

20. The system of reducing sugar intake of claim 8, wherein the evaluating the behavioral response comprises the collection of data concerning: a survey ranking a user's cravings for sugar; a survey of the user's diet; a survey of the user's mood changes; a survey of the user's adherence to the dietary schedule; and, the survey of perceived satisfaction with the sweetener monk fruit erythritol blend.
